(19) United States
(12) Patent Application Publication
(10) Pub. No.: US 2021/0251559 A1
(43) Pub. Date: Aug. 19, 2021

(54) METHODS FOR DIAGNOSING, MONITORING AND TREATING NEUROLOGICAL DISEASES AND DISORDERS

(71) Applicant: CuraSen Therapeutics, Inc., San Carlos, CA (US)

(72) Inventor: Anthony P. Ford, San Carlos, CA (US)

(21) Appl. No.: 16/972,985

(22) PCT Filed: Jun. 14, 2019
(86) PCT No.: PCT/US19/37371

§ 371 Date: Dec. 7, 2020

Related U.S. Application Data
(60) Provisional application No. 62/685,244, filed on Jun. 14, 2018.
(60) Provisional application No. 62/686,654, filed on Jun. 18, 2018.
(60) Provisional application No. 62/825,619, filed on Mar. 28, 2019.

(51) Int. Cl. A61B 5/00; A61B 5/055

(52) U.S. Cl. CPC A61B 5/4088; A61B 5/055; A61B 5/0042

(57) ABSTRACT

In various aspects and embodiments provided are compositions and methods for identifying patients in need of improving cognition and/or treating a neurodegenerative disease in a patient and treating such patient.

### CLAIM OF PRIORITY

This application claims the benefit of U.S. Provisional Patent Application Ser. No. 62/685,244, filed on Jun. 14, 2018, 62/686,654, filed on Jun. 18, 2018, and 62/825,619, filed on Mar. 28, 2019. The entire contents of the foregoing are hereby incorporated by reference.

### FIELD

The present disclosure in various aspects and embodiments relates to compositions and methods for improving cognition and/or treating a neurodegenerative disease in a patient.

### BACKGROUND

United States Patent Application Publication Number 20130096126 discloses “a method for enhancing learning or memory of both in a mammal having impaired learning or memory or both from a neuro-degenerative disorder, which entails the step of administering at least one compound or a salt thereof which is a β1-adrenergic receptor agonist, partial agonist or receptor ligand in an amount effective to improve the learning or memory or both of said mammal.”

United States Patent Application Publication Number 20140235726 discloses “a method of improving cognition in a patient with Down syndrome, which entails administering one or more β2 adrenergic receptor agonists to the patient in an amount and with a frequency effective to improve cognition of the patient as measured by contextual learning tests.”

United States Patent Application Publication Number 20160184241 discloses “a method of improving cognition in a patient with Down syndrome, which entails intranasally administering one or more β2-ADR agonists or pharmaceutically-acceptable salts of either or both to the patient in an amount and with a frequency effective to improve cognition of the patient as measured contextual learning tests.”

PCT Application Publication Number WO2017115873 discloses “a combination of two or more compounds selected from the group consisting of compounds represented by the Compound No. 1-130, a preventive or therapeutic agent for Alzheimer's disease (AD)” and states “In an attempt to achieve the aforementioned object, the present inventors have screened an existing drug library consisting of 1280 kinds of pharmaceutical compounds approved by the Food and Drug Administration (FDA) in America by using nerve cells induced to differentiate from iPS cells derived from AD patients, and extracted 129 kinds (including one kind of concomitant drug) of compounds that improve Aβ pathology in the nerve cells as candidate therapeutic drugs for AD.”

PCT Application Publication Number WO2006108424 states “[t]he invention furthermore relates to dermatological compositions without skin sensitization properties and which contain enantiomerically pure or enriched R-enantiomers of a β2 adrenoceptor agonist.

PCT Application Publication Number WO2018195473 provides “methods of treating a subject who has a synucleinopathy (e.g., Parkinson's disease) that include administering to a subject in need of such treatment therapeutically effective amounts of a β2-adrenoreceptor agonist and at least one therapeutic agent.”

### SUMMARY

In one aspect, a method is provided wherein the method includes subjecting a patient to brain imaging to determine regional metabolic activation in forebrain, midbrain and brainstem areas and/or to identify whether said patient is in need of or desiring improvement of cognitive function and/or treatment of a neurodegenerative disease; and subsequently administering to said patient a β1-ADR agonist and a peripherally acting β-blocker (PABRA) to improve cognition and/or treat a neurodegenerative disease in said patient. In some embodiments, the brain imaging is fluorodeoxyglucose positron emission tomography (FDG-PET), used alone or in combination with other imaging approaches such as magnetic resonance imaging (MRI) and CT. In some embodiments, the brain imaging is, or can include, magnetic resonance imaging-arterial spin labeling (MRI-ASL), or magnetic resonance imaging-blood oxygenation level dependent computerized tomography (MRI-BOLD). In some embodiments of the aspects and embodiments disclosed herein, “improving cognition and/or treating a neurodegenerative disease” in a patient may include improving cognitive and executive function, improving inflammatory status in cerebral or cerebrospinal fluid (CSF) samples, attenuating proteinopathies burden (for example, based on imaging or CSF sampling) and/or improving regional cerebral metabolic status (reversing hypometabolism) in the patient. Likewise, in certain embodiments, “identifying a patient in need of or desiring improvement of cognitive function and/or treatment of a neurodegenerative disease” may include identifying a patient in need of or desiring improvement of cognitive and executive function, improvement of inflammatory status in cerebral or CSF samples, attenuation of proteinopathies burden (for example, based on imaging or CSF sampling) and/or improvement of regional cerebral metabolic status (reversing hypometabolism). In another aspect, a method is provided wherein the method includes subjecting a patient to brain imaging to determine regional metabolic activation in forebrain, midbrain and brainstem areas and/or to identify whether said patient is in need of or desiring improvement of cognitive function and/or treatment of a neurodegenerative disease; administering to said patient a β1-ADR agonist and a peripherally acting β-blocker (PABRA) to improve cognition and/or treat a neurodegenerative disease in said patient; and subsequently re-subjecting said patient to brain imaging to determine any improvement in regional metabolic activation in forebrain, midbrain and brainstem areas, cognitive function and/or treatment of said neurodegenerative disease. In some embodiments, the brain imaging is FDG-PET, used alone or in combination with other imaging approaches such as MRI and CT. In some embodiments, the brain imaging is, or can include, MRI-ASL or MRI-BOLD. In yet another aspect, a method is provided wherein the method includes subjecting a patient to brain imaging determine regional metabolic activation in forebrain, midbrain and brainstem areas; administering to said patient a β1-ADR agonist and a peripherally acting β-blocker (PABRA); and subsequently re-subjecting said patient to brain imaging to determine any improvement in regional metabolic activation in forebrain, midbrain and brainstem areas, cognitive function. In some embodiments, the brain imaging is FDG-PET, used alone or in combination with other imaging approaches such as MRI and CT. In some embodiments, the brain imaging is, or can include, MRI-ASL or MRI-BOLD.

In some embodiments, a detectable label is provided, which can generate a spatial pattern of the brain imaging result. In some embodiments, 2-[¹⁸F]fluoro-2-deoxy-D-glucose (¹⁸FDG) can be used for FDG-PET, which can provide characteristic spatial patterns of brain metabolism and can help clinicians to make a reasonably accurate and early diagnosis for appropriate management or prognosis.

In some aspects, a method is provided wherein the method includes subjecting a patient to brain imaging to determine regional metabolic activation in forebrain, midbrain and brainstem areas and/or to identify whether said patient is in need of or desiring improvement of cognitive function and/or treatment of a neurodegenerative disease; and subsequently administering to said patient a β2-ADR agonist and a peripherally acting β-blocker (PABRA) to improve cognition and/or treat a neurodegenerative disease in said patient. In some embodiments, the brain imaging is fluorodeoxyglucose positron emission tomography (FDG-PET), used alone or in combination with other imaging approaches such as magnetic resonance imaging (MRI) and CT. In some embodiments, the brain imaging is, or can include, MRI-ASL or MRI-BOLD. In some embodiments of the aspects and embodiments disclosed herein, “improving cognition and/or treating a neurodegenerative disease” in a patient may include improving cognitive and executive function, improving inflammatory status in cerebral or cerebrospinal fluid (CSF) samples, attenuating proteinopathies burden (for example, based on imaging or CSF sampling) and/or improving regional cerebral metabolic status (reversing hypometabolism) in the patient. Likewise, in certain embodiments, “identifying a patient in need of or desiring improvement of cognitive function and/or treatment of a neurodegenerative disease” may include identifying a patient in need of or desiring improvement of cognitive and executive function, improvement of inflammatory status in cerebral or CSF samples, attenuation of proteinopathies burden (for example, based on imaging or CSF sampling) and/or improvement of regional cerebral metabolic status (reversing hypometabolism). In another aspect, a method is provided wherein the method includes subjecting a patient to brain imaging to determine regional metabolic activation in forebrain, midbrain and brainstem areas and/or to identify whether said patient is in need of or desiring improvement of cognitive function and/or treatment of a neurodegenerative disease; administering to said patient a β2-ADR agonist and a peripherally acting β-blocker (PABRA) to improve cognition and/or treat a neurodegenerative disease in said patient; and subsequently re-subjecting said patient to brain imaging to determine any improvement in regional metabolic activation in forebrain, midbrain and brainstem areas, cognitive function and/or treatment of said neurodegenerative disease. In some embodiments, the brain imaging is FDG-PET, used alone or in combination with other imaging approaches such as MRI and CT. In some embodiments, the brain imaging is, or can include, MRI-ASL or MRI-BOLD. In yet another aspect, a method is provided wherein the method includes subjecting a patient to brain imaging determine regional metabolic activation in forebrain, midbrain and brainstem areas; administering to said patient a β2-ADR agonist and a peripherally acting β-blocker (PABRA); and subsequently re-subjecting said patient to brain imaging to determine any improvement in regional metabolic activation in forebrain, midbrain and brainstem areas, cognitive function. In some embodiments, the brain imaging is FDG-PET, used alone or in combination with other imaging approaches such as MRI and CT. In some embodiments, the brain imaging is, or can include, MRI-ASL or MRI-BOLD. In some embodiments, the patient does not have Alzheimer's disease. In some embodiments, the patient does not have Down Syndrome. In some embodiments, the patient does not have Parkinson's disease. In some embodiments, the patient does not have dementia with Lewy bodies. In some embodiments, the β2-ADR agonist can be administered at a dose of from about 0.1 μg/kg to 1.5 g/kg of the patient's body weight. In some embodiments, the β2-ADR agonist can be administered at a dose of from about 1 μg/kg to 100 mg/kg of the patient's body weight.

In one aspect, a method is provided wherein the method includes subjecting a patient to brain imaging to determine regional metabolic activation in forebrain, midbrain and brainstem areas and/or to identify whether said patient is in need of or desiring improvement of cognitive function and/or treatment of a neurodegenerative disease; and subsequently administering to said patient prenalterol and optionally a peripherally acting β-blocker (PABRA) to improve cognition and/or treat a neurodegenerative disease in said patient. In some embodiments, the brain imaging is fluorodeoxyglucose positron emission tomography (FDG-PET), used alone or in combination with other imaging approaches such as magnetic resonance imaging (MRI) and CT. In some embodiments, the brain imaging is, or can include, MRI-ASL or MRI-BOLD. In some embodiments of the aspects and embodiments disclosed herein, “improving cognition and/or treating a neurodegenerative disease” in a patient may include improving cognitive and executive function, improving inflammatory status in cerebral or cerebrospinal fluid (CSF) samples, attenuating proteinopathies burden (for example, based on imaging or CSF sampling) and/or improving regional cerebral metabolic status (reversing hypometabolism) in the patient. Likewise, in certain embodiments, “identifying a patient in need of or desiring improvement of cognitive function and/or treatment of a neurodegenerative disease” may include identifying a patient in need of or desiring improvement of cognitive and executive function, improvement of inflammatory status in cerebral or CSF samples, attenuation of proteinopathies burden (for example, based on imaging or CSF sampling) and/or improvement of regional cerebral metabolic status (reversing hypometabolism). In another aspect, a method is provided wherein the method includes subjecting a patient to brain imaging to determine regional metabolic activation in forebrain, midbrain and brainstem areas and/or to identify whether said patient is in need of or desiring improvement of cognitive function and/or treatment of a neurodegenerative disease; administering to said patient prenalterol and optionally a peripherally acting β-blocker (PABRA) to improve cognition and/or treat a neurodegenerative disease in said patient; and subsequently re-subjecting said patient to brain imaging to determine any improvement in regional metabolic activation in forebrain, midbrain and brainstem areas, cognitive function and/or treatment of said neurodegenerative disease. In some embodiments, the brain imaging is FDG-PET, used alone or in combination with other imaging approaches such as MRI and CT. In some embodiments, the brain imaging is, or can include, MRI-ASL or MRI-BOLD. In yet another aspect, a method is provided wherein the method includes subjecting a patient to brain imaging determine regional metabolic activation in forebrain, midbrain and brainstem areas; administering to said patient prenalterol and optionally a peripherally acting β-blocker (PABRA); and subsequently re-subjecting said patient to brain imaging to determine any improvement in regional metabolic activation in forebrain, midbrain and brainstem areas, cognitive function. In some embodiments, the brain imaging is FDG-PET, used alone or in combination with other imaging approaches such as MRI and CT. In some embodiments, the brain imaging is, or can include, MRI-ASL or MRI-BOLD.

Prenalterol is a β1 agonist having the following chemical structure:

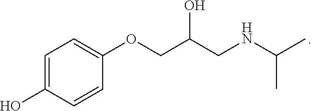 [SMILES: CC(C)NCC(O)COc1ccc(O)cc1]

In certain embodiments, prenalterol as used herein refers to a racemic mixture. In other embodiments, the prenalterol used herein may be (S)-prenalterol that is substantially free of the (R)-prenalterol isomer. In other embodiments, the prenalterol used herein may be (R)-prenalterol that is substantially free of the (S)-prenalterol isomer.

In one aspect, a method is provided wherein the method includes subjecting a patient to brain imaging to determine regional metabolic activation in forebrain, midbrain and brainstem areas and/or to identify whether said patient is in need of or desiring improvement of cognitive function and/or treatment of a neurodegenerative disease; and subsequently administering to said patient tulobuterol and optionally a peripherally acting β-blocker (PABRA) to improve cognition and/or treat a neurodegenerative disease in said patient. In some embodiments, the brain imaging is fluorodeoxyglucose positron emission tomography (FDG-PET), used alone or in combination with other imaging approaches such as magnetic resonance imaging (MRI) and CT. In some embodiments, the brain imaging is, or can include, MRI-ASL or MRI-BOLD. In some embodiments of the aspects and embodiments disclosed herein, “improving cognition and/or treating a neurodegenerative disease” in a patient may include improving cognitive and executive function, improving inflammatory status in cerebral or cerebrospinal fluid (CSF) samples, attenuating proteinopathies burden (for example, based on imaging or CSF sampling) and/or improving regional cerebral metabolic status (reversing hypometabolism) in the patient. Likewise, in certain embodiments, “identifying a patient in need of or desiring improvement of cognitive function and/or treatment of a neurodegenerative disease” may include identifying a patient in need of or desiring improvement of cognitive and executive function, improvement of inflammatory status in cerebral or CSF samples, attenuation of proteinopathies burden (for example, based on imaging or CSF sampling) and/or improvement of regional cerebral metabolic status (reversing hypometabolism). In another aspect, a method is provided wherein the method includes subjecting a patient to brain imaging to determine regional metabolic activation in forebrain, midbrain and brainstem areas and/or to identify whether said patient is in need of or desiring improvement of cognitive function and/or treatment of a neurodegenerative disease; administering to said patient tulobuterol and optionally a peripherally acting β-blocker (PABRA) to improve cognition and/or treat a neurodegenerative disease in said patient; and subsequently re-subjecting said patient to brain imaging to determine any improvement in regional metabolic activation in forebrain, midbrain and brainstem areas, cognitive function and/or treatment of said neurodegenerative disease. In some embodiments, the brain imaging is FDG-PET, used alone or in combination with other imaging approaches such as MRI and CT. In some embodiments, the brain imaging is, or can include, MRI-ASL or MRI-BOLD. In yet another aspect, a method is provided wherein the method includes subjecting a patient to brain imaging determine regional metabolic activation in forebrain, midbrain and brainstem areas; administering to said patient tulobuterol and optionally a peripherally acting β-blocker (PABRA); and subsequently re-subjecting said patient to brain imaging to determine any improvement in regional metabolic activation in forebrain, midbrain and brainstem areas, cognitive function. In some embodiments, the brain imaging is FDG-PET, used alone or in combination with other imaging approaches such as MRI and CT. In some embodiments, the brain imaging is, or can include, MRI-ASL or MRI-BOLD.

Tulobuterol is a long-acting β2 agonist having the following chemical structure:

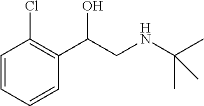

Tulobuterol is marketed in Japan as a racemic mixture for administration as a transdermal patch. In certain embodiments, tulobuterol as used herein refers to a racemic mixture. In other embodiments, the tulobuterol used herein may be (S)-tulobuterol that is substantially free of the (R)-tulobuterol isomer. In other embodiments, the tulobuterol used herein may be (R)-tulobuterol that is substantially free of the (S)-tulobuterol isomer.

In some aspects, a method is provided which includes treating a subject identified as having diminished cognitive function and/or being in need of or desiring improvement of cognitive function and/or treatment of a neurodegenerative disease by administering the subject a pharmaceutical composition including a β1-ADR agonist, a β2-ADR agonist, a peripherally acting β-blocker (PABRA), or any combination thereof. In some embodiments, the method further includes assessing effectiveness of the treatment. In some embodiments, the treatment is assessed by subjecting the subject to a test to assess improved cognitive function or amelioration of the neurodegenerative disease. In some embodiments, the method further includes adjusting administration of the pharmaceutical composition by adjusting dosage of the pharmaceutical composition and/or timing of administration of the pharmaceutical composition.

In some embodiments, the pharmaceutical composition includes a β1-ADR agonist and a PABRA. In some embodiments, the pharmaceutical composition includes a β2-ADR agonist and a PABRA. In some embodiments, the pharmaceutical composition includes prenalterol and optionally a PABRA. In some embodiments, the pharmaceutical composition includes tulobuterol and optionally a PABRA. In some embodiments, the β2-ADR agonist can be administered at a dose of from about 0.1 μg/kg to 1.5 g/kg of the patient's body weight. In some embodiments, the β2-ADR agonist can be administered at a dose of from about 1 μg/kg to 100 mg/kg of the patient's body weight.

As used herein, the term “β1 agonist” is used to mean β1-adrenergic receptor agonist or β1-ADR agonist. In certain embodiments the term β1 agonist is understood to include compounds that are primarily β1 agonists, but which may also exhibit some peripheral agonism for other adrenergic receptors, such as β2-adrenergic receptors. In this application, the terms “β1-adrenergic receptor agonist”, “β1-ADR agonist”, “β1AR agonist” and “β1 agonist” may be used interchangeably. In certain embodiments, the term β1-ADR agonist expressly includes both selective and partial agonists, as well as biased and non-biased agonists. Examples of β1 adrenergic agonists include, for example, xamoterol, noradrenalin, isoprenaline, dopamine, pindolol and dobutamine and the pharmaceutically-acceptable salts of any of the above. Partial agonists and ligands of the β1-ADR are known. Further, using the methodology of Kolb et al., but for β1-ADR instead, one skilled in the art could determine new ligands by structure-based discovery. See Proc. Natl. Acad. Sci. USA 2009, 106, 6843-648.

As used herein, the term “β2 agonist” is used to mean β2-adrenergic receptor agonist or β2-ADR agonist. In certain embodiments, the term β2 agonist is understood to include compounds that are primarily β2 agonists, but which may also exhibit some peripheral agonism for other adrenergic receptors, such as β1-adrenergic receptors. In this application the terms “β2-adrenergic receptor agonist”, “β2-ADR agonist”, “β2AR agonist” and “β2 agonist” may be used interchangeably. In some embodiments the term β2-ADR agonist expressly includes both selective and partial agonists. β2 agonists that may be used in accordance with various aspects and embodiments of the present disclosure may be short-acting, long-acting or ultra long-acting. Examples of short-acting β2 agonists that may be used are salbutamol, levo salbutamol, terbutaline, pirbuterol, procaterol, metaproterenol, bitolterol mesylate, oritodrine, isoprenaline, salmefamol, fenoterol, terbutaline, albuterol, and isoetharine. Examples of long-acting β2 agonists that may be used are salmeterol, bambuterol, formoterol and clenbuterol. Examples of ultra long-acting β2 agonists include indacaterol, vilanterol and olodaterol. Other examples of β2 agonists include tulobuterol, mabuterol, and ritodrine.

As used herein, the term “peripherally acting β-blocker (PABRA)” means a β adrenergic receptor antagonist or simply a β1-, β2- or non-selective β-blocker. Examples of selective peripherally acting β-blockers (PABRA) that may in certain embodiments be used in the methods disclosed herein include nadolol, atenolol, sotalol and labetalol. In certain embodiments a β-blocker that can be used in the methods herein is one or more selected from the group consisting of acebutolol, betaxolol, bisoprolol, celiprolol, esmolol, metaprolol and nevivolol; in other embodiments the methods do not use acebutolol, betaxolol, bisoprolol, celiprolol, esmolol, metaprolol or nevivolol as a β-blocker.

In some embodiments xamoterol and clenbuterol with or without nadolol are administered to the patient. In some embodiments xamoterol and formoterol with or without nadolol are administered to the patient. In some embodiments xamoterol and albuterol with or without nadolol are administered to the patient.

In certain embodiments a peripherally acting β-blocker (PABRA) is administered to the patient prior to administration of a β1-ADR agonist, a β2-ADR agonist, prenalterol, and/or tulobuterol; in other embodiments a peripherally acting β-blocker (PABRA) is administered to the patient concurrently with the administration of a β1-ADR agonist, a β2-ADR agonist, prenalterol, and/or tulobuterol.

In certain embodiments of the compositions and methods provided herein, one or more peripherally acting β-blocker (PABRA) are administered prior to or concurrently with a β1-ADR agonist, a β2-ADR agonist, prenalterol, and/or tulobuterol in order to inhibit or preclude agonism of peripheral β1 and/or β2 adrenergic receptors by the β1-ADR agonist, β2-ADR agonist, prenalterol, and/or tulobuterol. In various embodiments it is preferred to block peripheral β1 and/or β2 adrenergic receptors in accordance with the compositions and methods of the present disclosure in order to preclude, or at least minimize, any adverse peripheral cardiac effects on humans being treated.

In certain embodiments of the methods provided herein, the β1-ADR agonist, β2-ADR agonist, prenalterol, and/or tulobuterol is administered orally, intravenously, intramuscularly, by inhalation or intranasally. In certain embodiments of the methods provided herein, the β1-ADR agonist, β2-ADR agonist, prenalterol, and/or tulobuterol is administered orally. In certain embodiments of the methods provided herein, the β1-ADR agonist, a β2-ADR agonist, prenalterol, and/or tulobuterol is administered intranasally. In certain embodiments of the methods provided herein, the β1-ADR agonist, β2-ADR agonist, prenalterol, and/or tulobuterol is administered by inhalation.

In certain embodiments of the methods provided herein, the peripherally acting β-blocker (PABRA), if administered, is administered orally, intravenously, intramuscularly, by inhalation or intranasally. In certain embodiments of the methods provided herein, the peripherally acting β-blocker (PABRA), if administered, is administered orally. In certain embodiments of the methods provided herein, the peripherally acting β-blocker (PABRA), if administered, is administered by inhalation.

In certain embodiments of the methods provided herein a β1-ADR agonist, a β2-ADR agonist, prenalterol, or tulobuterol is administered to the patient intranasally and a peripherally acting β-blocker (PABRA) is administered peripherally (e.g, orally, intravenously, intramuscularly, or by inhalation).

In certain embodiments of the methods provided herein a β1-ADR agonist, a β2-ADR agonist, prenalterol, and/or tulobuterol and the peripherally acting β-blocker (PABRA) are administered to the patient orally. In certain embodiments of the methods provided herein β1-ADR agonist, β2-ADR agonist, prenalterol, and/or tulobuterol and the peripherally acting β-blocker (PABRA) are administered to the patient orally and both agents are present in a tablet.

In some embodiments of the aspects and embodiments provided herein, the patient is identified as having a neurodegenerative disease that is one or more selected from the group consisting of MCI (mild cognitive impairment), aMCI (amnestic MCI), Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson's disease), PDD (PD dementia), ADHD (attention deficit hyperactivity disorder), Alzheimer's disease (AD), early AD, and Down Syndrome (DS). In some embodiments the of the patient is identified as having a neurodegenerative disease that is one or more selected from the group consisting of MCI, aMCI, Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson's disease), PDD (PD dementia), and ADHD (attention deficit hyperactivity disorder). In some embodiments the patient does not have Alzheimer's disease (AD). In some embodiments the patient does not have Down Syndrome. In some embodiments the patient does not have Parkinson's disease. In some embodiments the patient does not have dementia with Lewy bodies.

In some embodiments the patient is subjected to a cognition test or model after said administration. In some embodiments the patient is subjected to a cognition test or model after said administration wherein the cognition test or model is a memory test; a diagnostic indicator of mental status, brain function, mental condition; a contextual learning test and/or brain imaging. In some embodiments the patient is subjected to a cognition test or model before said administration. In some embodiments the patient is subjected to a cognition test or model before said administration wherein the cognition test or model is a memory test; a diagnostic indicator of mental status, brain function, mental condition; a contextual learning test and/or brain imaging. In some embodiments the patient is subjected to a cognition test or model such as a memory test; a diagnostic indicator of mental status, brain function, mental condition; a contextual learning test and/or brain imaging before said administration and the cognition test or model is used to identify a patient in need of or desiring improvement of cognitive function and/or treatment of a neurodegenerative disease in accordance with the methods and compositions provided herein. In some embodiments the patient is subjected to a cognition test or model before and after said administration. In some embodiments the patient is subjected to a cognition test or model before and after said administration wherein the cognition test or model is a memory test; a diagnostic indicator of mental status, brain function, mental condition; a contextual learning test and/or brain imaging.

In certain embodiments, the patient demonstrates improved cognition following said administration. In some embodiments the patient demonstrates improved cognition as demonstrated by an improvement in a cognition test or model; a memory test; a diagnostic indicator of mental status, brain function, mental condition; a contextual learning test; brain imaging or the like in the patient.

“Improving cognition,” “improved cognition” or “improvement in cognition” means an improvement in an individual's cognitive capacity, or memory, or the like. In certain embodiments, the methods described herein result in an improvement cognition, for example as demonstrated by an improvement in a cognition test, a memory test, brain imaging and/or a contextual learning test in the patient. In some embodiments, the methods described herein result in an improvement in a contextual learning test in the patient wherein said contextual learning test is a spatial contextual learning test or Arizona Cognitive Test Battery (ACTB).

In some embodiments the patient is a mammal. In some embodiments the patient is a human. In some embodiments the patient is a child human. In some embodiments the patient is an adult human. Child, as used herein, means a human from about 5 to 20 years of age. Adult, as used herein, means a human from about 21 years of age and older.

### DETAILED DESCRIPTION OF SPECIFIC EMBODIMENTS

In certain aspects and embodiments of the present disclosure compositions and methods result in an improved cognition, raised cerebral metabolic activity and/or improved inflammatory control in a patient. In some embodiments the methods described herein result in an improvement cognition, for example as demonstrated by an improvement in a cognition test or model; a memory test; a diagnostic indicator of mental status, brain function, mental condition; a contextual learning test; or the like in the patient. Such cognitive tests, diagnostics and models are well known in the art. In various aspects and embodiments, any of many accepted contextual learning tests for animals or humans can be used to assess baseline cognitive function and/or to measure or quantify improved cognitive function. In some embodiments, the compositions and methods described herein may result in an improvement one or more tests, diagnostics and models as follows. Likewise for the raised cerebral metabolic activity and improved inflammatory control—these in certain embodiments may be imaged via FDG-PET and via sampling of cerebrospinal fluid (CSF) allowing measures of inflammatory cytokines and markers of glial cell activation. In some embodiments, magnetic resonance imaging-arterial spin labeling (MRI-ASL) can be used for neuroimaging. In some embodiments, magnetic resonance imaging-blood oxygenation level dependent computerized tomography (MRI-BOLD) can be used for neuroimaging. In various embodiments, FDG-PET may be used alone or in combination with CT and/or MRI including MRI-ASL and/or MRI-BOLD. For example, FDG-PET and MRI-BOLD may be used, or FDG-PET and MRI-ASL may be used. Alternatively, FDG-PET, MRI-BOLD and MRI-ASL may be used. Alternatively, MRI, including MRI-BOLD and MRI-ASL, may be used alone or in combination, and optionally with CT.

Animal Models/Tests

In some embodiments, brain imaging adapted for animal models may be used to monitor cognitive function and/or evaluate the effectiveness of treatments such as described herein. In some embodiments, fluorodeoxyglucose positron emission tomography (FDG-PET) may be used for neuroimaging, for example as described in Mirbolooki et al., Synapse, (2015) 69:96-98 and Catus et al., British Journal of Pharmacology, (2011) 162:1700-1715; see also Brown et al., RadioGraphics, (2014) 34:684-701, and Shivamurthy et al., A J R, (2015) 204:W76-W85.

Exploratory Activity in Novel Environment. The Activity Chamber (Med associates Inc., St. Albans, Vt.) may be used for the evaluation of general activity, gross locomotor activity, and exploratory behavior. Assessment may take place in a square arena, 43.2×43.2 cm, with 3 planes of infrared detectors within a specially designed sound attenuating chamber, 66×55.9×55.9 cm, under dim light. The animal is placed in the center of the testing arena and allowed to move freely for 30 minutes while being tracked by an automated tracking system. Distance moved, velocity, resting time, and vertical count (rearing) are recorded.

PhenoTyper. PhenoTyper® (Noldus Information Technology, Wageningen, the Netherlands) is an automated infrared video-based observation system for the measurement of behavior of mice in their home cage (see de Visser et al., 2006). The home cage environment minimizes stress or discomfort, and the subjects are given ad libitum access to all accessories in the PhenoTyper® chamber. PhenoTypers®, each containing one mouse, may be connected to a computer running Ethovision® XT (Noldus Information Technology, Wageningen, the Netherlands), which can acquire data over extended periods of time. After 3 days of baseline activity recording, a running wheel is placed in each cage. Distance moved, velocity of ambulatory movement and time spent in the shelter, food zone, water zone, and running wheel are measured during the experiment and reported separately for dark and light cycles.

Cat Walk. The CatWalk® apparatus (Noldus Information Technology, Wageningen, the Netherlands) consists of a glass floor illuminated with beams of fluorescent light. Assessment in a dark room allows the paws to reflect light as they come in contact with the glass floor. The bright pixel images are recorded by a camera directly below the glass walkway and digitally converted. The paw pixels are identified and analyzed by a blind observer, generating gait-related measurements (Starkey et al., 2005). Using home cage motivation, mice are trained to traverse the CatWalk® apparatus 1 day prior to gait assessment. Training assures that the animals walk consistently across the walkway without hesitation or exploratory behavior. On testing day, mice are given 3 consecutive runs, returning to their home cage each time. Runs in which an animal takes more than 8 seconds to cross the end zone, walks backwards, walks in the reverse direction, or rear are excluded, and the animal is allowed to run again. The average of 3 runs for each animal are reported. For this study, general gait parameters (regularity index, stride pattern, and running duration) as well as individual paw parameters (intensity, paw area, stand duration, and stride length) are analyzed.

Spontaneous Alternation. Spontaneous alternations are measured using the Y-maze and T-maze. The Y-shaped maze is constructed with 3 symmetrical white solid plastic arms at a 120° angle (40 cm length, 8 cm width, and 15 cm height). Each session begins with placement of the mouse in the center of the maze. The mouse is allowed to freely explore the 3 arms for 8 minutes. Arm entry is defined as all 4 limbs within the arm. The maze is cleaned with 10% ethanol between sessions to eliminate odor traces. The number of arm entries and the number of triads are recorded in order to calculate the alternation percentage, which is generated by dividing the number of triads by the number of possible alternations×100. A triad is defined as a set of arm entries, when each entry is to a different arm of the maze.

The T-maze has 3 equal arms (30 cm length, 10 cm width, and 20 cm height). The start arm and 2 goal arms have guillotine gates. This test is based on the rodents' preference to experience a new arm of the T-maze instead of a familiar one (Gerlai, 1998). In each trial, the mouse is placed in the start arm. The gate is then opened and the mouse is able to freely explore the arms. As soon as the mouse enters one goal arm, the sliding gate of the other goal arm is closed. The mouse eventually returns to the start arm and the next trial is started. In the next trial, the mouse may recognize the previously chosen goal arm and choose to explore a new arm rather than revisit the previously visited arm. This trial is repeated 11 times per day for 3 consecutive days, for a total of 33 trials. The maze is cleaned with 50% ethanol between trials to eliminate odor. Percent of alternation (number of turns in each goal arm) is used for analysis. This protocol has been described before by Belichenko et al. (2009) and is modified from a Deacon and Rawlins (2006) protocol (Deacon and Rawlins, 2006; Belichenko et al., 2009).

Intellicage. Home cage-based learning behaviors of socially housed mice are tested using the Intellicage® apparatus (NewBehavior AG, Zurich, Switzerland). Intellicage® is an automated home cage-based system for the evaluation of place and operant learning (see Galsworthy et al., 2005; Knapska et al., 2006 for details. Animals are randomly assigned to Intellicages® with 6-10 mice in each cage. The subjects are socially housed in these groups prior to the experiment. Forty eight hours before introduction into the Intellicage®, each animal is anesthetized by inhalation of isoflurane and injected subcutaneously with an RFID transponder (Datamars SA, Bedano, Switzerland). After general habituation to the cage, animals are subjected to the nose poke adaptation in order to learn to access the water during 2 drinking sessions every 24 hrs. Following these adaptation periods, the animals are subjected to 3 different tests: place learning, place avoidance, and entry to the novel satellite box. In the place learning test, each mouse has access to water in only one corner of the cage and learns to associate access to water with this specific corner of the cage for 4 consecutive days. In all trials, percent of correct visits during drinking sessions is reported. Following the place learning session, all animals are removed front the Intellicage®. This session is followed by a 72-hr delay before all animals are returned to the Intellicage® for the probe trial to evaluate the total number of visits to the correct corner. In the “place avoidance test,” animals learn to avoid a corner where they are met with the aversive stimulus of an air puff. After a 4-day training session, mice are removed from the apparatus for 72 hrs and then returned to the Intellicage® for a probe trial. During the probe trial, the animals receive no air puffs. The percentage of visits to the previously punished corner versus all corners is reported as the percent of incorrect visits (errors) for each day. In the novelty exploration test, prior to housing of animals in the Intellicage®, a smaller satellite box is attached with the entrance blocked on the end closest to the Intellicage®. The mice have access to water in all corners. Then the tunnel plug is removed and the animals are allowed to freely explore the novel satellite box. The latency to the first entrance to the satellite box and visit frequency is reported.

Delayed-Matched-To-Place Water Maze. The Delayed Match-To-Place (DMP) water maze task may be used to assess learning and memory as originally designed by Steel and Morris (Steele and Morris, 1999) for rats. Subjects are given a series of 4 trials approximately 8-10 min apart in a large water tank (178 cm in diameter) filled with opaque water at a temperature of 22.0±1.5° C. A 15 cm circular platform is submerged 1 cm below the water surface and placed randomly in the pool with daily changes in position. The release point in the pool is changed based on the experimental set up. Each animal is given a maximum of 90 seconds to find the submerged platform. If they are unable to find the platform in that time, the animals are physically guided to it. After remaining on the platform for 10 seconds, the animals are removed and placed in a dry cage. This process is repeated for 7 days. After training on DMP, subjects are given visible platform training to ensure they have no gross sensorimotor or visual deficit. During visible platform training, the platform is marked with a black and white ping-pong bail attached to a 10 cm wooden stick. The swim paths of the animals are recorded with the Ethovision® 3.1 computer-interfaced camera tracking system (Noldus Information Technology, Wageningen, the Netherlands) and subsequently analyzed. The water is frequently changed and the tank disinfected.

Fear Conditioning and Startle Response Tests. Contextual and Cued fear conditioning is conducted for evaluation of fear-dependent learning and retrieval in the study. The test is performed using chambers from Coulbourn Instruments (Whitehall, Pa.). On the first day, the animals are placed in a chamber (Context A) for 3 min for baseline recording, followed by 5 tone-shock pairings. The shock (0.5 mA, 2 sec) is delivered following the tone (70 dB, 2 kHz, 20 sec) in each conditional/unconditional stimulus pairing. On the second day a novel chamber (Context B; new room, new olfactory environment, texture of floor, blue plastic inserts for walls, extra source of blue light, and visual cues) is used for cued testing. Three tones without shocks are presented to animals during a 3 min testing period following a 3 min pre-tone period. On the last day of the experiment, the mice are placed in Context A for 5 min without any conditional and unconditional stimulus (modified from the method described by Saxe et al., 2006). Freezing is defined as the complete lack of motion for a minimum of 0.75 second as measured by FreezeFrame® software (Actimetrics, Evanston, Ill.). The percent of freezing in each period is reported. For the startle response control test, an acoustic startle reflex apparatus (Med Associates Inc., St. Albans, Vt.) is used. The subjects are acclimated to the animal holder in the startle box for a total of 15 min over 3 consecutive-days prior to the experiment. The animals are exposed to 25 different trials with 10-20 second randomly variable inter-trial intervals. Five different intensities of startle pulses, 0, 90, 100, 110, and 120 dB, are randomly used, and each animal is randomly exposed 5 times to each intensity of the startle pulse. The duration of each startle pulse is 40 msec and the peak amplitude of the startle response in each trial is recorded for analysis. The holding cage on the apparatus is cleaned with 10% alcohol between each animal.

Three-Chambered Sociability and Social Novelty Test. An established three-chambered box test (Moy et al., 2004; Crawley, 2007; Moy et al., 2007) may be used to assess sociability and interest in social novelty. Before testing, object mice are habituated to a pencil cup 10 mm per day over 3 consecutive days. Between subjects, the box and pencil cup are cleaned with paper towels and diluted ethanol. Testing consists of three 10-minute sessions, in the first “habituation” session, subject mice are freely allowed to investigate the three-chambered box. This is followed by a “sociability” session where a never-before-met C57B1/6J male mouse is placed in one of the pencil cups. The location of the stranger mouse is alternated from left to right across subject testing. In the “social novelty” session, a second never-before-met C57B1/6J male mouse is placed under the second pencil cup. Trials are video recorded for subsequent rating. Measured parameters are number of entrances into the chambers, time spent in chambers, and time spent sniffing the pencil cups.

Social Memory Testing. Prior to social memory testing, randomly selected individually housed ovariectomized C57B1/6J female mice (OEF) are put into the home cages of subject mice 4 hrs per day for 5-7 days to reduce sexual behavior.

Two-Trial Jest: A never-before-met OEF is placed into the home cage of a test animal for 5 rain and then removed. After an inter-trial interval (ITI) of 30 mm, the same OEF is placed back in the home cage together with a novel never-before-met OEF for 5 mm. Trials are videotaped and analyzed as in the five-trial social memory test.

Five-Trial Test: A single OEF is introduced into the home cage of a never-before-met test animal for four 1-min exposures with an inter-trial interval of 10 min. In a fifth trial 10 min later, instead of the familiar OEF, a novel, never-before-met OEF is put into the home cage of the test animal for 1 min. All trials are videotaped and subsequently analyzed for olfactory investigation. Investigation is defined as nose-to-body contact of the test animal versus the intruder. Total investigation, including ano-genital investigation, perioral investigation, and body investigation are measured in two 30-second bins.

Olfactory Habituation Test. The test may consist of 2-min presentations of 6 different cotton swabs soaked with 100 μL of liquid separated by 3-min ITI's. The tip of the cotton swab is placed 1 cm above the bedding in the home cage to allow investigation without rearing. After 3 presentations of distilled water, the animals receive 3 presentations of either pure urine from a never-before-met singly housed OEF mouse or almond scent (1:100 in distilled water). Trials are videotaped for subsequent scoring. Direct physical contact between the nose and the cotton swab was scored; chewing the cotton swab is excluded.

Human Models/Tests

There are many contextual learning tests used that are acknowledged and/or accepted in the art that in various embodiments may be used in conjunction with the compositions and methods disclosed herein to assess baseline cognitive function and/or to measure or quantify improved cognitive function in human subjects. For example, the contextual learning test used may be based upon single task learning, multiple task learning or spatial contextual memory. Contextual learning test evaluations based upon spatial contextual memory may be advantageous in assessing, for example, how well an individual is able to navigate a shopping mall, his or her neighborhood or a city transit or subway system as well as assessing any improvements in the ability to execute these tasks resulting from the treatment methods described herein.

An example of a simple spatial contextual learning test is contextual cuing, where humans learn to use repeated spatial configurations to facilitate a target search. A higher order spatial contextual learning test is serial learning, where humans learn to use subtle sequence regularities to respond more quickly and accurately to a series of events. See, for example, J. H. Howard Jr., et al., Neuropsychology, Vol. 18(1), January 2004, 124-134.

In some embodiments, cognition may be evaluated using the Mini-Mental State Examination (MMSE) and/or the Montreal Cognitive Assessment (MOCA).

Arizona Cognitive Test Battery (ACTB). A testing protocol that may be used in various embodiments is the Arizona Cognitive Test Battery (ACTB). See Edgin, J., et al. J. Neurodevelop. Disord. (2010) 2: 149-164. The ACTB has been developed specifically to assess the cognitive phenotype in DS, and includes various tests with various task demands and links with brain function. In more detail, tests are included for: 1) benchmarks, such as KBIT II verbal subscale and KBIT II non-verbal subscale IQ tests, 2) hippocampal function, 3) prefrontal function, 4) cerebellar function, 5) Finger sequencing tasks, 6) NEPSY visuomotor precision and 7) simple reaction time.

A correlation of domain/test, test description and primary ability assessed in accordance with the ACTB is provided below:

| | | Primary Ability |
|---|---|---|
| Domain/Test | Description | Assessed |
| 1) Benchmark | Points to pictures based on word or | Verbal comprehension |
| KBIT-II verbal subscale | phrase | Problem solving |
| KBIT-II nonverbal subscale | Semantic or visuo-spatial | |
| | pattern completion | |
| 2) CANTAB spatial span | Touching boxes in order of | Immediate memory for |
| | changing color on screen | spatial-temporal sequence |
| 3) Prefrontal Modified dots | Press button below a cat, shifts to | Inhibitory control |
| task | new rule, press across screen for a | working memory |
| | frog, etc. | |
| 4) CANTAB IED | Forced-choice discrimination task | Set-shifting |
| | with change in relevant dimension | |
| 5) Hippocampal CANTAB | Recall for hidden abstract patterns | Spatial associative |
| paired associates | | memory |
| 6) Virtual computer- | Navigation of a virtual arena(via | Spatial memory |
| generated arena | joystick) to find a hidden target | |
| 7) Cerebellar | Sequences generated by tapping a | Motor sequencing |
| Finger-sequencing task | number of fingers (1, 2, 3, 4) to a | |
| | lever in succession | |
| 8) NEPSY visuo-motor | Follows two tracks with a pen | Visuo-motor tracking, |
| precision | | hand-eye coord. |
| 9) CANTAB simple reaction | Participants press button in | Motor response time and |
| time | response to a box presented on a | attention |
| | screen | |

The above battery of tests in some embodiments may all be performed in order to assess all major cognitive processes balanced by the practical need for testing under time constraints. The cognitive tests herein may in certain embodiments be used in patients receiving treatment herein to monitor the patient's cognitive status and progression.

In some embodiments, the battery of tests may be conducted with a test group of individuals, and a control group individuals to demonstrate the effectiveness of various aspects and embodiments of the compositions and methods described herein. The test group may be treated with any of the treatment regimens described herein, and the control group is treated with placebo, such as a dextrose 5% saline solution by intranasal administration.

An improvement in cognitive function as defined herein as being at least a 10%, and preferably at least a 20% score improvement, on at least one, and preferably two or more, of the tests listed in the ATCB, for example. Anyone of the domain/tests listed for the ATCB above may be included in assessing whether an improvement occurred. Testing may be conducted after treatment or during treatment to ascertain whether modifications in dosage or frequency of treatment is warranted.

Brain Imaging. Generally, any non-invasive procedure many be used to both establish a baseline of brain pathology (existent or non-existent) from which baseline a treatment protocol is established. However, magnetic resonance imaging (MRI) may in some embodiments be preferred for neuroimaging examination because it allows for accurate measurement of the 3-dimensional (3D) volume of brain structures, especially the hippocampus and related regions. Such techniques are well known as described in U.S. Pat. No. 6,490,472, which patent is incorporated herein in the entirety.

Moreover, non-invasive optical imaging systems may also be used for monitoring neurological pathological events. See, for example, U.S. patent publication 2011/0286932, which is incorporated herein in the entirety. The technique described therein entails administration of a fluorescent marker to a human for staining Aβ peptides, imaging the retina of the DS human with an optical imaging system, and examining the images for stained Aβ peptides in order to determine whether onset of brain pathology (such as AD brain pathology) has occurred.

In certain embodiments, fluorodeoxyglucose positron emission tomography (FDG-PET) may be used for neuroimaging to determine cognitive function and/or identify a neurodegenerative disease in accordance with the compositions and methods described herein. The use of FDG-PET for monitoring cognitive function and/or diagnosing cognitive impairments or neurodegenerative diseases, and/or identifying patients in need of or desiring a treatment to improve cognitive function is described in, for example Brown et al., RadioGraphics, (2014) 34:684-701, and Shivamurthy et al., AJR, (2015) 204:W76-W85; both hereby incorporated by reference in their entirety. In various embodiments, FDG-PET may be used alone or in combination with CT and/or MRI including MRI-ASL and/or MRI-BOLD. For example, FDG-PET and MRI-BOLD may be used, or FDG-PET and MRI-ASL may be used. Alternatively, FDG-PET, MRI-BOLD and MRI-ASL may be used. Alternatively, MRI, including MRI-BOLD and MRI-ASL, may be used alone or in combination, and optionally with CT.

Alzheimer's Disease

AD brain pathology refers to the accumulation of highly degradation-resistant amyloid fibers that cause lesions in areas of the brain proximate thereto. Accumulation of these amyloid fibers to neurotoxic levels leads to destruction of nerve fibers, which, in turn, leads to the observed behavior associated with Alzheimer's dementia. Observed behavioral symptoms, which become progressively more severe with progression of the disease, often include loss of vocabulary, incorrect word substitutions (paraphasias), loss of reading and writing skills, increased risk of falling, wandering, loss of speech, apathy and even loss of muscle mass.

Down Syndrome

Creation of several trisomic mouse models has greatly facilitated progress in the understanding the neurobiological basis of cognitive dysfunction in DS. Among the mouse models, the Ts65Dn mouse is best characterized. It has an extra copy of approximately 140 mouse genes on chromosome 16, orthologous to those on human chromosome 21 (HSA21). Almost all genes in HSA21 with potential role in nervous system abnormalities are also found in Ts65Dn mice Similar to DS, alterations in the structure and function of the hippocampus and failure in the induction of long-term potentiation (LTP) have been extensively reported in Ts65Dn mice. Ts65Dn mice are the most widely used in DS research, and are considered to be an art-accepted model for investigations regarding DS in humans. Olson, L. E., et al., Dev. Dyn. 2004 July; 230(3):581-9.

DS is characterized by degeneration and dysfunction of multiple neuronal populations in the central nervous system (CNS). Among them, the hippocampal formation, i.e. the primary site for processing contextual learning shows significant abnormalities in DS. As a result, failure in contextual learning is a common finding in people with DS. To uncover the neurobiological basis of failed contextual learning in DS, the integrity of subcortical regions extensively projecting to the hippocampal formation have been examined. Through extensive innervation, these subcortical regions impose strong modulatory influence on hippocampal neurons. Among these subcortical regions, LC is of particular importance. LC neurons in the brainstem are the sole supplier of massive norepinephrine (NE)-ergic terminals for the hippocampus and play a significant role in wakefulness, attention, and navigational memory. Significant age-related degeneration of NE-ergic neurons of LC in Ts65Dn mice was found. Interestingly, the loss of LC terminals in Ts65Dn mice leads to further deterioration of cognitive dysfunction in these mice. Similarly, LC neurons undergo extensive age-dependent degeneration in DS. The critical role of NE-ergic system dysfunction in cognitive dysfunction in Ts65Dn has been supported by the fact that increasing brain NE levels with L-threo-3, 4-dihydroxyphenylserine (L-DOPS), i.e. a NE prodrug, restored contextual learning in Ts65Dn mice. Although L-DOPS is in phase III clinical trial for the treatment of primary autonomic failure associated with Parkinson's disease, it is yet to be approved by the FDA and its long-term effects particularly in children have yet to be explored.

With respect to the agents described herein, the terms “modulate” and “modulation” refers to the upregulation (i.e., activation or stimulation) or downregulation (i.e., inhibition or suppression) of a response. A “modulator” is an agent, compound, or molecule that modulates, and may be, for example, an agonist, antagonist, activator, stimulator, suppressor, or inhibitor. The terms “inhibit”, “reduce”, remove as used herein refer to any inhibition, reduction, decrease, suppression, downregulation, or prevention in expression, activity or symptom and include partial or complete inhibition of activity or symptom. Partial inhibition can imply a level of expression, activity or symptom that is, for example, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% of the uninhibited expression, activity or symptom. The terms “eliminate” or “eradicate” indicate a complete reduction of activity or symptom.

As used herein, the term “a disorder” or “a disease” refers to any derangement or abnormality of function; a morbid physical or mental state. See Dorland's Illustrated Medical Dictionary, (W.B. Saunders Co. 27th ed. 1988).

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

In some embodiments, optically pure (S)-β agonist is used to the extent the β2 agonist has a stereocenter, which is substantially free of (R)-β agonist. In some embodiments, optically pure (R)-β agonist is used, which is substantially free of (S)-β agonist. The term “pure”, as used herein, refers to substances that have been separated from at least some or most of the components with which they are associated in nature or when originally generated or with which they were associated prior to purification. In general, such purification involves action of the hand of man. Pure agents may be partially purified, substantially purified, or pure. Such agents may be, for example, at least 50%, 60%, 70%, 75%, 80%, 85%, 90%, 95%, 96%, 97%, 98%, 99%, or more than 99% pure. In some embodiments, a nucleic acid, polypeptide, or small molecule is purified such that it constitutes at least 75%, 80%, 85%, 90%, 95%, 96%, 97%, 98%, 99%, or more, of the total nucleic acid, polypeptide, or small molecule material, respectively, present in a preparation. In some embodiments, an organic substance, e.g., a nucleic acid, polypeptide, or small molecule, is purified such that it constitutes at least 75%, 80%, 85%, 90%, 95%, 96%, 97%, 98%, 99%, or more, of the total organic material present in a preparation. Purity may be based on, e.g., dry weight, size of peaks on a chromatography tracing (GC, HPLC, etc.), molecular abundance, electrophoretic methods, intensity of bands on a gel, spectroscopic data (e.g., NMR), elemental analysis, high throughput sequencing, mass spectrometry, or any art-accepted quantification method. In some embodiments, water, buffer substances, ions, and/or small molecules (e.g., synthetic precursors such as nucleotides or amino acids), can optionally be present in a purified preparation. A purified agent may be prepared by separating it from other substances (e.g., other cellular materials), or by producing it in such a manner to achieve a desired degree of purity.

In some embodiments, contemplated methods may include for example, administering prodrugs of the compounds described herein, or a pharmaceutical composition thereof. The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al., Nature Reviews Drug Discovery 2008, 7, 255). In some embodiments, the prodrug structures are constructed according to the disclosure in U.S. Pat. No. 9,849,134, which is incorporated by reference herein in the entirety.

For example, if a compound of the disclosure or a pharmaceutically acceptable salt, hydrate or solvate of the compound contains a carboxylic acid functional group, a prodrug can comprise an ester formed by the replacement of the hydrogen atom of the acid group with a group such as (C₁₋₈)alkyl, (C₂₋₁₂)alkylcarbonyloxymethyl, 1-(alkylcarbonyloxy)ethyl having from 4 to 9 carbon atoms, 1-methyl-1-(alkylcarbonyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁₋₂)alkylamino-(C₂₋₃)alkyl (such as β-dimethylaminoethyl), carbamoyl-(C₁₋₂)alkyl, N,N-di(C₁₋₂)alkylcarbamoyl-(C₁₋₂)alkyl and piperidino-, pyrrolidino- or morpholino(C₂₋₃)alkyl.

Similarly, if a compound of the disclosure contains an alcohol functional group, a prodrug can be formed by the replacement of the hydrogen atom of the alcohol group with a group such as (C₁₋₆)alkylcarbonyloxymethyl, 1-((C₁₋₆)alkylcarbonyloxy)ethyl, 1-methyl-1-((C₁₋₆)alkylcarbonyloxy)ethyl (C₁₋₆)alkoxycarbonyloxy)methyl, N—(C₁₋₆)alkoxycarbonylaminomethyl, succinoyl, (C₁₋₆)alkylcarbonyl, α-amino(C₁₋₄)alkylcarbonyl, arylalkylcarbonyl and α-aminoalkylcarbonyl, or α-aminoalkylcarbonyl α-aminoalkylcarbonyl, where each α-aminoalkylcarbonyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

If a compound of the disclosure incorporates an amine functional group, a prodrug can be formed, for example, by creation of an amide or carbamate, an N-alkylcarbonyloxyalkyl derivative, an (oxodioxolenyl)methyl derivative, an N-Mannich base, imine or enamine. In addition, a secondary amine can be metabolically cleaved to generate a bioactive primary amine, or a tertiary amine can metabolically cleaved to generate a bioactive primary or secondary amine. For examples, see Simplicio, et al., Molecules 2008, 13, 519 and references therein.

“Therapeutically effective amount” as used herein means the amount of a compound or composition (such as described herein) that causes at least one desirable change in a cell, population of cells, tissue, individual, patient or the like. In some embodiments a therapeutically effective amount as used herein means the amount of a compound or composition (such as described herein) that prevents or provides a clinically significant change in a disease or condition (e.g., reduce by at least about 30 percent, at least about 50 percent, or at least about 90 percent) or in one or more features of a disease or condition described herein. In some embodiments, the term “therapeutically effective amount” means an amount of a compound or composition as described herein effective or sufficient to improve cognition and/or treat a neurodegenerative disease in a patient. The term “frequency” as related thereto means the number of times a treatment is administered to a patient in order to obtain the result of improved cognition and/or treating a neurodegenerative disease in a patient.

Diagnostics and Assessment of Treatment

In various aspects, the methods of the disclosure include diagnosing or otherwise identifying whether a patient is in need of or desiring improvement of cognitive function and/or treatment of a neurodegenerative disease. As discussed herein, this may be performed in a variety of ways as discussed herein and generally known in the art. For example, a patient diagnosis may be made by brain imaging. In various embodiments, FDG-PET may be used alone or in combination with CT and/or MRI including MRI-ASL and/or MRI-BOLD. For example, FDG-PET and MRI-BOLD may be used, or FDG-PET and MRI-ASL may be used. Alternatively, FDG-PET, MRI-BOLD and MRI-ASL may be used. Alternatively, MRI, including MRI-BOLD and MRI-ASL, may be used alone or in combination, and optionally with CT.

Along with identifying suitable patients for treatment, diagnosis allows further determinations to be made regarding various aspects of the type and mode of treatment to be administered. For example, depending on the diagnosis, determinations may be made regarding the pharmaceutical active to be administered, the dosage of such actives as well as the timing schedule of administration.

A diagnostic method utilized with the methods of the disclosure may make use of a detectable label to diagnose or otherwise identify a patient that is in need of or desiring improvement of cognitive function and/or treatment of a neurodegenerative disease. The term “label” (also referred to as “detectable label”) refers to any moiety that facilitates detection and, optionally, quantification, of an entity that comprises it or to which it is attached. The label can be conjugated to or otherwise attached to a variety of entities, biological or otherwise. In general, a label may be detectable by, e.g., spectroscopic, photochemical, biochemical, immunochemical, electrical, optical, chemical or other means. In some embodiments a detectable label produces an optically detectable signal (e.g., emission and/or absorption of light), which can be detected e.g., visually or using suitable instrumentation such as a light microscope, a spectrophotometer, a fluorescence microscope, a fluorescent sample reader, a fluorescence activated cell sorter, a camera, or any device containing a photodetector. Labels that may be used in various embodiments include, e.g., organic materials (including organic small molecule fluorophores (sometimes termed “dyes”), quenchers (e.g., dark quenchers), polymers, fluorescent proteins); enzymes; inorganic materials such as metal chelates, metal particles, colloidal metal, metal and semiconductor nanocrystals (e.g., quantum dots); compounds that exhibit luminescence upon enzyme-catalyzed oxidation such as naturally occurring or synthetic luciferins (e.g., firefly luciferin or coelenterazine and structurally related compounds); haptens (e.g., biotin, dinitrophenyl, digoxigenin); radioactive atoms (e.g., radioisotopes such as ³H, ¹⁴C, ³²P, ³³P, ³⁵S, ¹²⁵I), stable isotopes (e.g., ¹³C, ²H); magnetic or paramagnetic molecules or particles, and the like. Fluorescent dyes include, e.g., acridine dyes; BODIPY, coumarins, cyanine dyes, napthalenes (e.g., dansyl chloride, dansyl amide), xanthene dyes (e.g., fluorescein, rhodamines), and derivatives of any of the foregoing. Examples of fluorescent dyes include Cy3, Cy3.5, Cy5, Cy5.5, Cy7, Alexa® Fluor dyes, DyLight® Fluor dyes, FITC, TAMRA, Oregon Green dyes, Texas Red, to name but a few. Fluorescent proteins include green fluorescent protein (GFP), blue, sapphire, yellow, red, orange, and cyan fluorescent proteins and fluorescent variants such as enhanced GFP (eGFP), mFruits such as mCherry, mTomato, mStrawberry; R-Phycoerythrin, and the like. Enzymes useful as labels include, e.g., enzymes that act on a substrate to produce a colored, fluorescent, or luminescent substance. Examples include luciferases, β-galactosidase, horseradish peroxidase, and alkaline phosphatase. Luciferases include those from various insects (e.g., fireflies, beetles) and marine organisms (e.g., cnidaria such as Renilla (e.g., Renilla reniformis, copepods such as Gaussia (e.g., Gaussia princeps) or Metridia (e.g., Metridia longa, Metridia pacifica), and modified versions of the naturally occurring proteins. A wide variety of systems for labeling and/or detecting labels or labeled entities are known in the art. Numerous detectable labels and methods for their use, detection, modification, and/or incorporation into or conjugation (e.g., covalent or noncovalent attachment) to biomolecules such as nucleic acids or proteins, and the like, are described in lain Johnson, I., and Spence, M. T. Z. (Eds.), The Molecular Probes® Handbook—A Guide to Fluorescent Probes and Labeling Technologies. 11th edition (Life Technologies/Invitrogen Corp.) available online on the Life Technologies website at invitrogen.com/site/us/en/home/References/Molecular-Probes-The-Handbook.html and Hermanson, G T., Bioconjugate Techniques, 2^{nd} ed., Academic Press (2008). Many labels are available as derivatives that are attached to or incorporate a reactive functional group so that the label can be conveniently conjugated to a biomolecule or other entity of interest that comprises an appropriate second functional group (which second functional group may either occur naturally in the biomolecule or may be introduced during or after synthesis). For example, an active ester (e.g., a succinimidyl ester), carboxylate, isothiocyanate, or hydrazine group can be reacted with an amino group; a carbodiimide can be reacted with a carboxyl group; a maleimide, iodoacetamide, or alkyl bromide (e.g., methyl bromide) can be reacted with a thiol (sulfhydryl); an alkyne can be reacted with an azide (via a click chemistry reaction such as a copper-catalyzed or copper-free azide-alkyne cycloaddition). Thus, for example, an N-hydroxysuccinide (NHS)-functionalized derivative of a fluorophore or hapten (such as biotin) can be reacted with a primary amine such as that present in a lysine side chain in a protein or in an aminoallyl-modified nucleotide incorporated into a nucleic acid during synthesis. A label may be directly attached to an entity or may be attached to an entity via a spacer or linking group, e.g., an alkyl, alkylene, aminoallyl, aminoalkynyl, or oligoethylene glycol spacer or linking group, which may have a length of, e.g., between 1 and 4, 4-8, 8-12, 12-20 atoms, or more in various embodiments. A label or labeled entity may be directly detectable or indirectly detectable in various embodiments. A label or labeling moiety may be directly detectable (i.e., it does not require any further reaction or reagent to be detectable, e.g., a fluorophore is directly detectable) or it may be indirectly detectable (e.g., it is rendered detectable through reaction or binding with another entity that is detectable, e.g., a hapten is detectable by immunostaining after reaction with an appropriate antibody comprising a reporter such as a fluorophore or enzyme; an enzyme acts on a substrate to generate a directly detectable signal). A label may be used for a variety of purposes in addition to or instead of detecting a label or labeled entity. For example, a label can be used to isolate or purify a substance comprising the label or having the label attached thereto.

The term “labeled” is used herein to indicate that an entity (e.g., a molecule, such as a biological or small molecule, organic compound, probe, cell, tissue, and the like) comprises or is physically associated with (e.g., via a covalent bond or noncovalent association) a label, such that the entity can be detected. In some embodiments a detectable label is selected such that it generates a signal that can be measured and whose intensity is related to (e.g., proportional to) the amount of the label. In some embodiments two or more different labels or labeled entities are used or present in a composition. In some embodiments the labels may be selected to be distinguishable from each other. For example, they may absorb or emit light of different wavelengths. In some embodiments the labels may be selected to interact with each other. For example, a first label may be a donor molecule that transfers energy to a second label, which serves as an acceptor molecule through nonradiative dipole-dipole coupling as in resonance energy transfer (RET), e.g., Forster resonance energy transfer (FRET, also commonly called fluorescence resonance energy transfer).

Nuclear imaging is one of the most important tools of diagnostic medicine wherein an estimated 12-14 million nuclear medicine procedures are performed each year in the United States alone. Diagnostic nuclear imaging is therefore crucial for studies which determine the cause of a medical problem based on organ function, in contrast to radiographic studies, which determine the presence of disease based on static structural appearance.

Diagnostic radiopharmaceuticals and radiotracers are often designed or selected capable of selective binding to specific receptors by means of a binding moiety, such as an antibody, a specific inhibitor or other target-specific ligand. These targeted markers can therefore concentrate more rapidly in areas of interest, such as inflamed tissues, tumors, malfunctioning organs or an organ undergoing heightened expression of certain proteins. Thus, a blood circulating radiopharmaceutical is picked up by a specific organ or pathological tissue to a different extent than by other or non-pathological tissue. For example, a highly vascularized tissue (e.g., of a growing tumor) may concentrate more of a radiopharmaceutical while an ischemic tissue may concentrate less of the radiopharmaceutical than the surrounding tissues. Nuclear imaging relies on these general phenomena of varied distribution of radiopharmaceutical according to different tissue as well as different pathologies. As a result, specific tissue types (e.g., tumor tissues) may be distinguished from other tissues in radioactive-emission imaging.

Radiopharmaceuticals, which may be used in the process of differential diagnosis of pathologies may be conjugated to targeting (recognition binding) moieties and include a wide range of radioisotopes as mentioned below. Such radiopharmaceuticals therefore include recognition moieties such as, for example, monoclonal antibodies (which bind to a highly specific pre-determined target), fibrinogen (which is converted into fibrin during blood clotting), glucose and other chemical moieties and agents. Commonly used diagnostic conjugated radiopharmaceuticals include, for example, 2-[¹⁸F]fluoro-2-deoxy-D-glucose (¹⁸FDG), ¹¹¹In-Pentetreotide ([¹¹¹In-DTPA-D-Phe¹]-octreotide), L-3-[¹²³I]-Iodo-α-methyl-tyrosine (IMT), O-(2-[¹⁸F]fluoroethyl)-L-tyrosine (L-[¹⁸F]FET), ¹¹¹In-Capromab Pendetide (CYT-356, Prostascint) and ¹¹¹In-Satumomab Pendetide (Oncoscint).

Two basic techniques are widely used for nuclear imaging: positron emission tomography (PET) and single photon emission computed tomography (SPECT). PET detects photons generated through positron-electron annihilation of positrons from a diagnostic radiopharmaceutical tracer placed in the subject, e.g., patient, to be imaged, and analyzes the photon energy and trajectory to generate tomographic images of the patient. SPECT generates images by computer analysis of photon emission events from a diagnostic radiopharmaceutical tracer having gamma emitting isotopes. Both PET and SPECT require the detection and analysis of single photon events, which are characterized by low signal to noise ratio and scarcity relative to the background radiation. Other constraints on the PET and SPECT image qualities include the sensitivity, temporal and spatial resolution, dynamic range, response time and counting rate characteristics of the data acquisition probe devices, e.g., photomultipliers and the like.

Radioisotopes that emit both high energy γ and/or low energy γ, β and/or positron radiation and which can be used per se or as a part of a compound as radiopharmaceuticals, include, without limitation, technetium-99m (^{99m}Tc), gallium-67 (⁶⁷Ga), thallium-201 (²⁰¹Tl), 111 indium-(¹¹¹In), iodine-123 (¹²³I), iodine-125 (¹²⁵I), iodine-131 (¹³¹I), xenon-133 (¹³³Xe), and fluorine-18 (¹⁸F). All these isotopes, except ^{99m}Tc, ¹³¹I and ¹³³Xe, are produced in particle accelerators.

Non-limiting examples of commonly used radiotracers include ^{99m}Tc-Arcitumomab (CEA-Scan™) which is a monoclonal antibody for imaging colorectal tissues afflicted with colorectal cancer, ^{99m}Tc-sestamibi (Cardiolite™) and ^{99m}Tc-tetrofosmin (Myoview™) for imaging the heart of a subject for myocardial perfusion, ¹¹¹In-Capromab pendetide (ProstaScint™) which is a monoclonal antibody for imaging prostate tissues afflicted with prostate cancer, ^{99m}Tc-Fanolesomab (NeutroSpec™) which is a monoclonal antibody for imaging inflamed and infectious tissues and ⁹⁰Y/111In-Zevalin (Ibritumomab Tiuxetan) which is a monoclonal antibody directed against the CD20 antigen, whereby this antigen is found on the surface of normal and malignant B lymphocytes.

Any diagnostic radiopharmaceutical can be utilized in the kit of the present embodiments. Exemplary radiopharmaceuticals that can be utilized in this context of the present invention include, without limitation, ³H-water, ³H-inulin, ¹¹C-carbonmonoxide, ¹³N-ammonia, ¹⁴C-inulin, ¹⁵O—H₂O, ¹⁵O— O₂, ¹⁸F-fluorodeoxyglucose, ¹⁸F-sodium fluoride, ⁵¹Cr-erythrocytes (RBC), ⁵⁷Co-vitamin B12 (cyanocobalamin), ⁵⁸Co-vitamin B12 (cyanocobalamin), ⁵⁹Fe-citrate, ⁶⁰Co-vitamin B12 (cyanocobalamin), ⁶⁷Ga-citrate, ⁶⁸Ga-citrate, ⁷⁵Se-selenomethionine, ^{81m}Kr-krypton for inhalation, oral administration or injections, ⁸²Rb, ⁸⁵Sr-nitrate, ⁹⁰Y/¹¹¹In-ibritumomab tiuxetan (⁹⁰Y/¹¹¹In-Zevalin), ^{99m}Tc-albumin microspheres, ^{99m}Tc-disofenin, lidofenin and mebrofenin, ^{99m}Tc-DMSA, ^{99m}Tc-DTPA (injection), ^{99m}Tc-DTPA (aerosol), ^{99m}Tc-ECD (ethylene cystate dimer), ^{99m}Tc-exametazime (HMPAO), ^{99m}Tc-glucoheptonate, ^{99m}Tc-HEDP, ^{99m}Tc-HMDP, ^{99m}Tc-HSA, ^{99m}Tc-MAA, ^{99m}Tc-MAG.sub.3, ^{99m}Tc-MDP, ^{99m}Tc-tetrofosmin (Myoview), ^{99m}Tc-sestamibi (Cardiolite), ^{99m}Tc-oral administrations, ^{99m}Tc-pertechnetate, ^{99m}Tc-pyrophosphate, ^{99m}Tc-RBC in vitro and in vivo labeling, ^{99m}Tc-sulfur colloid, ^{99m}Tc-teboroxime, ^{99m}Tc-white blood cells, ¹¹¹In-ibritumomab tiuxetan (¹¹¹In-Zevalin), ¹¹¹In-DTPA, ¹¹¹In-platelets, ¹¹¹In-RBC, ¹¹¹In-white blood cells, ¹²³I-hippuran, ¹²³I-IMP, ¹²³I-mIBG, ¹²³I-sodium iodide, ¹²⁴I-sodium iodide, ¹²⁵I-fibrinogen, ¹²⁵I-IMP, ¹²⁵I-mIBG, ¹²⁵I-sodium iodide, ¹²⁶I-sodium iodide, ¹³⁰I-sodium iodide, ¹³¹I-hippuran, ¹³¹I-HSA, ¹³¹I-MAA, ¹³¹I-mIBG, ¹³¹I-Rose Bengal, ¹³¹I-sodium iodide, ¹²⁷Xe-inhalation and injection, ¹³³Xe-inhalation and injection, ¹⁹⁷Hg-chlormerodrin, ¹⁹⁸Au-colloid and ²⁰¹Tl-chloride.

The diagnostic methods described herein may also but utilized to assess the effectiveness of a particular therapeutic regimen. For example, a patient that has been identified as being in need of or desiring improvement of cognitive function and/or treatment of a neurodegenerative disease and which is being treated, may be diagnosed or otherwise assessed to determine the effectiveness of the treatment regime. While the diagnosis or assessment may be performed by any method known in the art, cognitive testing or brain imaging may be used to determine improvement of cognitive function or amelioration of a disease. In embodiments, cognitive testing or brain imaging may be used alone or in combination. In embodiments where brain imaging is utilized, FDG-PET may be used alone or in combination with CT and/or MRI including MRI-ASL and/or MRI-BOLD. For example, FDG-PET and MRI-BOLD may be used, or FDG-PET and MRI-ASL may be used. Alternatively, FDG-PET, MRI-BOLD and MRI-ASL may be used. Alternatively, MRI, including MRI-BOLD and MRI-ASL, may be used alone or in combination, and optionally with CT.

The assessment of treatment efficacy may be utilized to alter the treatment regime of a patient. For example, the assessment may be utilized to alter dosing, timing of administration, and/or the actives of the pharmaceutical composition. In embodiments, the dosage of a particular pharmaceutical agent being administered to the patient may be lowered by combining administration with a different agent. In this manner, treatment may be optimized by altering the pharmaceutical composition to include different combinations of β1-ADR agonist, β2-ADR agonist, and peripherally acting β-blocker (PABRA). Dosing may also be altered depending on the timing of administration. For example, a shorter duration between each administration of the pharmaceutical composition may require a lower dose of active agent, while a longer duration between each administration of the pharmaceutical composition may require a higher dose of active agent, either of which may improve the treatment regime as determined by diagnosis or assessment of the patient.

In one embodiment, a patient may be assessed a single time during the course of treatment to optimize the treatment regime. Alternatively, the patient may be assessed multiple times over the course of treatment to continually optimize the treatment regime as directed by a medical professional.

Dosage, Administration and Pharmaceutical Formulation

The term “pharmaceutically-accepted salts” means acid addition salts that are commonly used in human or veterinary medicine and are deemed safe for use. Examples for the present disclosure include, but are not limited to, salts obtained from the following acids: acetic, ascorbic, benzenesulfonic, benzoic, camphosulfonic, citric, ethanesulfonic, edisylic, fumaric, gentisic, gluconic, glucoronic, glutamic, hippuric, hydrobromic, isethionic, lactic, nitric, phosphoric, succinic, sulfuric and tartaric, for example. Any hydrated forms of such salts are also included in this definition. Thus, for example, both fumarate and hemifumarate salts are specifically contemplated as well as any hydrates thereof. For example, fumarate dihydrate may be specifically mentioned.

The pharmaceutical preparation in some embodiments may be in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents. Preferred pharmaceutical preparations can deliver the compounds of the disclosure in a sustained release formulation.

For a binding agent, composition, or compound according to the present disclosure, the dosage form may optionally be a liquid dosage form. Solutions can be prepared in water suitably mixed with a surfactant such as hydroxypropylcellulose or an emulsifier such as polysorbate. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. Formulations optionally contain excipients including, but not limited to, a buffering agents, an anti-oxidant, a stabilizer, a carrier, a diluent, and an agent for pH adjustment. The pharmaceutical forms suitable for injectable use include sterile aqueous solutions or dispersion and sterile powders for the extemporaneous preparation of sterile injectable solutions or dispersions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl, or benzyl alcohol; alkyl parabens such as methyl or propyl paraben; catechol; resorcinol; cyclohexanol; 3-pentanol; and m-cresol); low molecular weight (less than about 10 residues) polypeptides; proteins such as serum, albumin, gelatin, or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS or polyethylene glycol (PEG).

In treatment, the dose of agent optionally ranges from about 0.0001 mg/kg to about 100 mg/kg, about 0.01 mg/kg to about 5 mg/kg, about 0.15 mg/kg to about 3 mg/kg, 0.5 mg/kg to about 2 mg/kg and about 1 mg/kg to about 2 mg/kg of the subject's body weight. In other embodiments the dose ranges from about 100 mg/kg to about 5 g/kg, about 500 mg/kg to about 2 mg/kg and about 750 mg/kg to about 1.5 g/kg of the subject's body weight. For example, depending on the type and severity of the disease, about 1 μg/kg to 15 mg/kg (e.g., 0.1-20 mg/kg) of agent is a candidate dosage for administration to the patient, whether, for example, by one or more separate administrations, or by continuous infusion. A typical daily dosage is in the range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. Unit doses can be in the range, for instance of about 5 mg to 500 mg, such as 50 mg, 100 mg, 150 mg, 200 mg, 250 mg and 300 mg. The progress of therapy is monitored by conventional techniques and assays.

In some embodiments, an agent is administered to a human patient at an effective amount (or dose) of less than about 1 μg/kg, for instance, about 0.35 to about 0.75 μg/kg or about 0.40 to about 0.60 μg/kg. In some embodiments, the dose of an agent is about 0.35 μg/kg, or about 0.40 μg/kg, or about 0.45 μg/kg, or about 0.50 μg/kg, or about 0.55 μg/kg, or about 0.60 μg/kg, or about 0.65 μg/kg, or about 0.70 μg/kg, or about 0.75 μg/kg, or about 0.80 μg/kg, or about 0.85 μg/kg, or about 0.90 μg/kg, or about 0.95 μg/kg or about 1 μg/kg. In various embodiments, the absolute dose of an agent is about 2 μg/subject to about 45 μg/subject, or about 5 to about 40, or about 10 to about 30, or about 15 to about 25 μg/subject. In some embodiments, the absolute dose of an agent is about 20μ.g, or about 30 μg, or about 40 μg.

In various embodiments, the dose of an agent may be determined by the human patient's body weight. For example, an absolute dose of an agent of about 2 μg for a pediatric human patient of about 0 to about 5 kg (e.g. about 0, or about 1, or about 2, or about 3, or about 4, or about 5 kg); or about 3 μg for a pediatric human patient of about 6 to about 8 kg (e.g. about 6, or about 7, or about 8 kg), or about 5 μg for a pediatric human patient of about 9 to about 13 kg (e.g. 9, or about 10, or about 11, or about 12, or about 13 kg); or about 8 μg for a pediatric human patient of about 14 to about 20 kg (e.g. about 14, or about 16, or about 18, or about 20 kg), or about 12 μg for a pediatric human patient of about 21 to about 30 kg (e.g. about 21, or about 23, or about 25, or about 27, or about 30 kg), or about 13 μg for a pediatric human patient of about 31 to about 33 kg (e.g. about 31, or about 32, or about 33 kg), or about 20 μg for an adult human patient of about 34 to about 50 kg (e.g. about 34, or about 36, or about 38, or about 40, or about 42, or about 44, or about 46, or about 48, or about 50 kg), or about 30 μg for an adult human patient of about 51 to about 75 kg (e.g. about 51, or about 55, or about 60, or about 65, or about 70, or about 75 kg), or about 45 μg for an adult human patient of greater than about 114 kg (e.g. about 114, or about 120, or about 130, or about 140, or about 150 kg).

In certain embodiments, an agent in accordance with the methods provided herein is administered subcutaneously (s.c.), intravenously (i.v.), intramuscularly (i.m.), intranasally or topically. Administration of an agent described herein can, independently, be one to four times daily or one to four times per month or one to six times per year or once every two, three, four or five years. Administration can be for the duration of one day or one month, two months, three months, six months, one year, two years, three years, and may even be for the life of the human patient. The dosage may be administered as a single dose or divided into multiple doses. In some embodiments, an agent is administered about 1 to about 3 times (e.g. 1, or 2 or 3 times).

### EXAMPLES

The present disclosure will be further described in the following examples, which do not limit the scope of the present disclosure.

### Example 1: Animal Models

Male and female Ts65Dn mice (B6EiC3Sn-al A-Ts (17₁₆)65Dn) and their age-matched normosomic (2N) littermates, aged 9-12 months, are used in this experiment. The genotype of all animals is determined by real-time quantitative PCR before starting the experiments. Because the retinal degeneration1 mutant gene (Rd1) is carried in the background of the mice, and this gene is recessive, mice homozygous for Rd1 are not used for the study. All animals are housed in a 12 hour dark/light cycle in a temperature- and humidity-controlled environment with ad libitum access to water and food; all tests are conducted in the light cycle.

Animals are divided in to treatment and control groups and are subjected to the one of the treatment regimens described herein. The animals are subjected to brain imaging and optionally one or more of the cognitive tests described herein before administration of the treatment regimen and again after the treatment or control regimen. Animals receiving the treatment regimen show improvements in the brain imaging and/or cognitive tests whereas control animals do not.

### Example 2: Treatment of Human Patients

A patient is screened using FDG-PET brain imaging. The identified as diagnosed with one or more of MCI, aMCI, Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson's disease), PDD (PD dementia), or ADHD (attention deficit hyperactivity disorder). Nadolol is administered to the patient followed by intranasal administration of the β1-ADR agonist, β2-ADR agonist, prenalterol, and/or tulobuterol. The treatment regimen is continued for one month. The patient is subjected to cognitive tests and FDG-PET imaging as described herein prior to commencement of the treatment regimen and approximately four weeks after the initiation of the treatment regimen. The patient demonstrates improvement in the cognitive tests following the treatment regimen and the FDG-PET imaging demonstrate improvement with regard to indica of brain disease. In some embodiments, magnetic resonance imaging-arterial spin labeling (MRI-ASL) can be used for neuroimaging. In some embodiments, magnetic resonance imaging-blood oxygenation level dependent computerized tomography (MRI-BOLD) can be used for neuroimaging.

### Example 3: Preparation of Substantially Free Prenalterol Stereoisomers

Optically pure (S)-prenalterol is prepared according to the following scheme using chemical synthesis methods that are well known in the art.

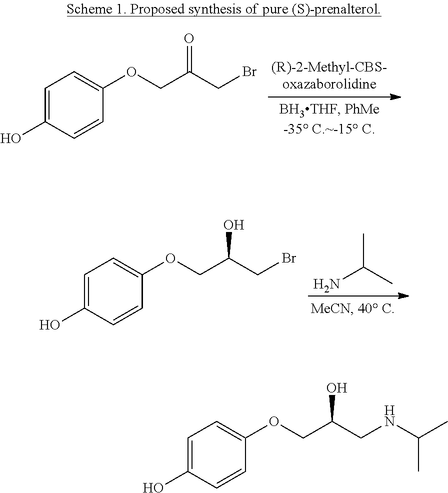

One ordinary skilled in the art can utilize routine purification technology such as HPLC or flash chromatography to purify the mixture from the above reaction to obtain optically pure (S)-prenalterol that is substantially free of (R)-prenalterol. Alternatively optically pure (S)-prenalterol can be isolated from a racemic mixture, for example by following procedures outlined in patent JP 54151935; or using routine chiral HPLC separation technology (Journal of Pharmaceutical and Biomedical Analysis, 2018, 70-81); and using SFC separation technology (Journal of Chromatography A, 2014, 85-97). In some embodiments, the phenol group can be protected before performing the proposed synthesis as outlined in Scheme 1.

Conversely, optically pure (R)-prenalterol that is substantially free of (5)-prenalterol is prepared according to the above scheme but replacing (R)-2-Methyl-CBS-oxazaborolidine in the scheme to (S)-2-Methyl-CBS-oxazaborolidine. Optically pure (R)-prenalterol that is substantially free of (5)-prenalterol can also be isolated from a racemic mixture using the above methodology to obtain optically pure (5)-prenalterol.

### Example 4: Preparation of Substantially Free Tulobuterol Stereoisomers

Optically pure (S)-tulobuterol is prepared according to the following scheme using chemical synthesis methods that are well known in the art.

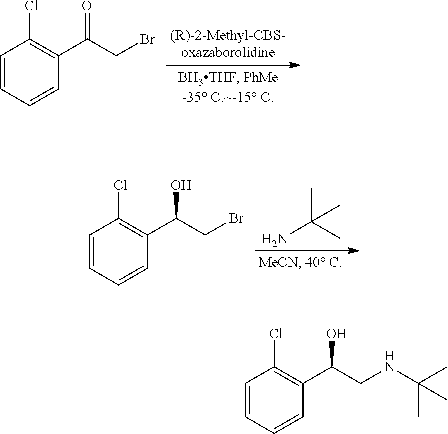

One ordinary skilled in the art can utilize routine purification technology such as HPLC or flash chromatography to purify the mixture from the above reaction to obtain optically pure (S)-tulobuterol that is substantially free of (R)-tulobuterol. Alternatively optically pure (s)-tulobuterol can be isolated from a racemic mixture, for example by following procedures outlined in patent JP 54151935; or using routine chiral HPLC separation technology (Journal of Pharmaceutical and Biomedical Analysis, 2018, 70-81); and using SFC separation technology (Journal of Chromatography A, 2014, 85-97).

Conversely, optically pure (R)-tulobuterol that is substantially free of (S)-tulobuterol is prepared according to the above scheme but replacing (R)-2-Methyl-CBS-oxazaborolidine in the scheme to (S)-2-Methyl-CBS-oxazaborolidine. Optically pure (R)-tulobuterol that is substantially free of (S)-tulobuterol can also be isolated from a racemic mixture using the above methodology to obtain optically pure (S)-tulobuterol.

### Aspects and Embodiments of the Disclosure

In one aspect, the disclosure provides a method that includes: subjecting a patient to brain imaging to determine cognitive function and/or to identify whether said patient is in need of or desiring improvement of cognitive function and/or treatment of a neurodegenerative disease; identifying a particular type of neurodegenerative disease based on a spatial pattern of the brain imaging result; and subsequently administering to said patient a β1-ADR agonist and a peripherally acting β-blocker (PABRA).

In another aspect, the disclosure provides a method including: subjecting a patient to brain imaging to determine cognitive function and/or to identify whether said patient is in need of or desiring improvement of cognitive function and/or treatment of a neurodegenerative disease; identifying a particular type of neurodegenerative disease based on a spatial pattern of the brain imaging result; administering to said patient a β1-ADR agonist and a peripherally acting β-blocker (PABRA) to improve cognition and/or treat a neurodegenerative disease in said patient; and subsequently re-subjecting said patient to brain imaging to determine any improvement in cognitive function and/or treatment of said neurodegenerative disease.

In yet another aspect, the disclosure provides a method including: subjecting a patient to brain imaging to determine cognitive function in said patient; identifying a particular type of neurodegenerative disease based on a spatial pattern of the brain imaging result; administering to said patient a β1-ADR agonist and a peripherally acting β-blocker (PABRA); and subsequently re-subjecting said patient to brain imaging to determine any improvement in cognitive function.

In still another aspect, the disclosure provides a method including: subjecting a patient to brain imaging to determine cognitive function and/or to identify whether said patient is in need of or desiring improvement of cognitive function and/or treatment of a neurodegenerative disease; identifying a particular type of neurodegenerative disease based on a spatial pattern of the brain imaging result; and subsequently administering to said patient a β2-ADR agonist and a peripherally acting β-blocker (PABRA).

In another aspect, the disclosure provides a method including: subjecting a patient to brain imaging to determine cognitive function and/or to identify whether said patient is in need of or desiring improvement of cognitive function and/or treatment of a neurodegenerative disease; identifying a particular type of neurodegenerative disease based on a spatial pattern of the brain imaging result; administering to said patient a β2-ADR agonist and a peripherally acting β-blocker (PABRA) to improve cognition and/or treat a neurodegenerative disease in said patient; and subsequently re-subjecting said patient to brain imaging to determine any improvement in cognitive function and/or treatment of said neurodegenerative disease.

In another aspect, the disclosure provides a method including: subjecting a patient to brain imaging to determine cognitive function in said patient; identifying a particular type of neurodegenerative disease based on a spatial pattern of the brain imaging result; administering to said patient a β2-ADR agonist and a peripherally acting β-blocker (PABRA); and subsequently re-subjecting said patient to brain imaging to determine any improvement in cognitive function.

In another aspect, the disclosure provides a method including: subjecting a patient to brain imaging to determine cognitive function and/or to identify whether said patient is in need of or desiring improvement of cognitive function and/or treatment of a neurodegenerative disease; identifying a particular type of neurodegenerative disease based on a spatial pattern of the brain imaging result; and subsequently administering to said patient prenalterol and a optionally peripherally acting β-blocker (PABRA).

In another aspect, the disclosure provides a method including: subjecting a patient to brain imaging to determine cognitive function and/or to identify whether said patient is in need of or desiring improvement of cognitive function and/or treatment of a neurodegenerative disease; identifying a particular type of neurodegenerative disease based on a spatial pattern of the brain imaging result; administering to said patient prenalterol and a optionally peripherally acting β-blocker (PABRA) to improve cognition and/or treat a neurodegenerative disease in said patient; and subsequently re-subjecting said patient to brain imaging to determine any improvement in cognitive function and/or treatment of said neurodegenerative disease.

In another aspect, the disclosure provides a method including: subjecting a patient to brain imaging to determine cognitive function in said patient; identifying a particular type of neurodegenerative disease based on a spatial pattern of the brain imaging result; administering to said patient prenalterol and a optionally peripherally acting β-blocker (PABRA); and subsequently re-subjecting said patient to brain imaging to determine any improvement in cognitive function.

In another aspect, the disclosure provides a method including: subjecting a patient to brain imaging to determine cognitive function and/or to identify whether said patient is in need of or desiring improvement of cognitive function and/or treatment of a neurodegenerative disease; identifying a particular type of neurodegenerative disease based on a spatial pattern of the brain imaging result; and subsequently administering to said patient tulobuterol and a optionally peripherally acting β-blocker (PABRA).

In another aspect, the disclosure provides a method including: subjecting a patient to brain imaging to determine cognitive function and/or to identify whether said patient is in need of or desiring improvement of cognitive function and/or treatment of a neurodegenerative disease; identifying a particular type of neurodegenerative disease based on a spatial pattern of the brain imaging result; administering to said patient tulobuterol and a optionally peripherally acting β-blocker (PABRA) to improve cognition and/or treat a neurodegenerative disease in said patient; and subsequently re-subjecting said patient to brain imaging to determine any improvement in cognitive function and/or treatment of said neurodegenerative disease.

In another aspect, the disclosure provides a method including: subjecting a patient to brain imaging to determine cognitive function in said patient; identifying a particular type of neurodegenerative disease based on a spatial pattern of the brain imaging result; administering to said patient tulobuterol and a optionally peripherally acting β-blocker (PABRA); and subsequently re-subjecting said patient to brain imaging to determine any improvement in cognitive function.

In another aspect, the disclosure provides a method including treating a subject identified as having diminished cognitive function and/or being in need of or desiring improvement of cognitive function and/or treatment of a neurodegenerative disease by administering the subject a pharmaceutical composition including a β1-ADR agonist, a β2-ADR agonist, a peripherally acting β-blocker (PABRA), or any combination thereof. In some embodiments, the method further includes assessing effectiveness of the treatment, the treatment can be assessed by subjecting the subject to a test to assess improved cognitive function or amelioration of the neurodegenerative disease. In some embodiments, the method further includes adjusting administration of the pharmaceutical composition by adjusting dosage of the pharmaceutical composition and/or timing of administration of the pharmaceutical composition.

In embodiments of any aspect or embodiment of the disclosure described herein, the brain imaging is fluorodeoxyglucose positron emission tomography (FDG-PET) scan, magnetic resonance imaging-arterial spin labeling (MRI-ASL), or magnetic resonance imaging-blood oxygenation level dependent computerized tomography (MRI-BOLD).

In embodiments of any aspect or embodiment of the disclosure described herein, said β1-ADR agonist is xamoterol or pindolol.

In embodiments of any aspect or embodiment of the disclosure described herein, said β2-ADR agonist is administered at a dose of from about 0.1 μg/kg to 1.5 g/kg of the patient's body weight.

In embodiments of any aspect or embodiment of the disclosure described herein, said β2-ADR agonist is administered at a dose of from about 1 μg/kg to 100 mg/kg of the patient's body weight.

In embodiments of any aspect or embodiment of the disclosure described herein, said β2-ADR agonist is one or more selected from the group consisting of mabuterol, ritodrine, salmeterol, bambuterol, formoterol and clenbuterol.

In embodiments of any aspect or embodiment of the disclosure described herein, said peripherally acting β-blocker (PABRA), if present, is one or more selected from the group consisting of nadolol, atenolol, sotalol and labetalol.

In embodiments of any aspect or embodiment of the disclosure described herein, said peripherally acting β-blocker (PABRA) is nadolol.

In embodiments of any aspect or embodiment of the disclosure described herein, said peripherally acting β-blocker (PABRA) is atenolol.

In embodiments of any aspect or embodiment of the disclosure described herein, said prenalterol, β1-ADR agonist, β2-ADR agonist and/or peripherally acting β-blocker (PABRA) are each administered orally.

In embodiments of any aspect or embodiment of the disclosure described herein, said prenalterol β1-ADR agonist, and/or β2-ADR agonist are administered intranasally.

In embodiments of any aspect or embodiment of the disclosure described herein, said prenalterol β1-ADR agonist, and/or β2-ADR agonist are administered intranasally and wherein said peripherally acting β-blocker (PABRA), if present, is administered peripherally (eg, orally, intravenously, or by inhalation).

In embodiments of any aspect or embodiment of the disclosure described herein, said neurodegenerative disease is one or more selected from MCI, aMCI, Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson's disease), PDD (PD dementia), ADHD (attention deficit hyperactivity disorder), and Down Syndrome.

In embodiments of any aspect or embodiment of the disclosure described herein, said neurodegenerative disease is one or more selected from MCI, aMCI, Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson's disease), PDD (PD dementia), and ADHD (attention deficit hyperactivity disorder).

In embodiments of any aspect or embodiment of the disclosure described herein, said patient does not have Alzheimer's disease.

In embodiments of any aspect or embodiment of the disclosure described herein, said patient does not have Down Syndrome.

In embodiments of any aspect or embodiment of the disclosure described herein, said patient does not have Parkinson's disease.

In embodiments of any aspect or embodiment of the disclosure described herein, said patient does not have dementia with Lewy bodies.

In embodiments of any aspect or embodiment of the disclosure described herein, said prenalterol is (S)-prenalterol that is substantially free of (R)-prenalterol.

In embodiments of any aspect or embodiment of the disclosure described herein, said prenalterol is (R)-prenalterol that is substantially free of (S)-prenalterol.

In embodiments of any aspect or embodiment of the disclosure described herein, said tulobuterol is (S)-tulobuterol that is substantially free of (R)-tulobuterol.

In embodiments of any aspect or embodiment of the disclosure described herein, said tulobuterol is (R)-tulobuterol that is substantially free of (S)-tulobuterol.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

All references referred to in the present disclosure are hereby incorporated by reference in their entirety. Various embodiments of the present disclosure may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

The embodiments of the disclosure described above are intended to be merely exemplary; numerous variations and modifications will be apparent to those skilled in the art. All such variations and modifications are intended to be within the scope of the present disclosure as defined in any appended claims.

1-67. (canceled)

68. A method comprising:
subjecting a patient to brain imaging to determine cognitive function and/or to identify whether said patient is in need of or desiring improvement of cognitive function and/or treatment of a neurodegenerative disease;
identifying a particular type of neurodegenerative disease based on a spatial pattern of the brain imaging result; and
subsequently administering to said patient a β2-ADR agonist and a peripherally acting β-blocker (PABRA).

69. The method of claim 68, further comprising: after administering to said patient said β2-ADR agonist re-subjecting said patient to brain imaging to determine any improvement in cognitive function and/or treatment of said neurodegenerative disease.

70. The method of claim 68, wherein said β2-ADR agonist is selected from the group consisting of salbutamol, levosalbutamol, terbutaline, pirbuterol, procaterol, metaproterenol, bitolterol mesylate, oritodrine, isoprenaline, salmefamol, fenoterol, terbutaline, albuterol, isoetharine, salmeterol, bambuterol, formoterol and clenbuterol, indacaterol, vilanterol and olodaterol, tulobuterol, mabuterol, and ritodrine.

71. The method of claim 68, wherein said β2-ADR agonist is clenbuterol.

72. The method of claim 68, wherein said β2-ADR agonist is tulobuterol.

73. The method of claim 68, wherein said PABRA is one or more selected from the group consisting of nadolol, atenolol, sotalol, labetalol, acebutolol, betaxolol, bisoprolol, celiprolol, esmolol, metaprolol and nevivolol.

74. The method of claim 68, wherein said PABRA is nadolol.

75. The method of claim 68, wherein said PABRA is labetalol.

76. The method of claim 68, wherein said neurodegenerative disease is one or more selected from the group consisting of MCI, aMCI, Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), AD (Alzheimer's Disease), FXS (fragile X syndrome), TSC (tuberous sclerosis complex), prion-related diseases (CJD etc.), depressive disorders, DLB (dementia with Lewy bodies), PD (Parkinson's disease), PDD (PD dementia), ADHD (attention deficit hyperactivity disorder), and Down Syndrome.

77. The method of claim 68, wherein said patient does not have Alzheimer's disease.

78. The method of claim 68, wherein said patient does not have Down Syndrome.

79. The method of claim 68, wherein said patient does not have Parkinson's disease.

80. The method of claim 68, wherein said patient does not have dementia with Lewy bodies.

81. The method of claim 68, wherein the test is brain imaging.

82. The method of claim 68, wherein the test is fluorodeoxyglucose positron emission tomography (FDG-PET) scan, magnetic resonance imaging-arterial spin labeling (Mill-ASL), or magnetic resonance imaging-blood oxygenation level dependent computerized tomography (MRI-BOLD).

83. The method of claim 68, wherein the test is fluorodeoxyglucose positron emission tomography (FDG-PET) scan.

84. The method of claim 68, wherein the test is magnetic resonance imaging-arterial spin labeling (MRI-ASL).

85. The method of claim 68, wherein the test is magnetic resonance imaging-blood oxygenation level dependent computerized tomography (MRI-BOLD).

86. The method of claim 68, wherein the dosage of the pharmaceutical composition is adjusted based on the test result.

87. A method comprising:
identifying a particular type of neurodegenerative disease based on a spatial pattern of brain imaging result in a subject; and subsequently administering to said patient a β2-ADR agonist and a peripherally acting β-blocker (PABRA) when the patient is in need of or desiring improvement of cognitive function and/or treatment of a neurodegenerative disease.
